(19) United States
(12) Patent Application Publication
(10) Pub. No.: US 2008/0027540 A1
(43) Pub. Date: Jan. 31, 2008

(54) STABILIZED ACCOMMODATING INTRAOCULAR LENS

(76) Inventor: J. Stuart Cumming, Laguna Beach, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 11/470,866

(22) Filed: Sep. 7, 2006

Related U.S. Application Data
(63) Continuation-in-part of application No. 11/461,290, filed on Jul. 31, 2006, now abandoned.

(51) Int. Cl. A61F 2/16

(52) U.S. Cl. 623/6.37; 623/6.44; 623/6.49

(57) ABSTRACT

An accommodating intraocular lens where the optic is moveable relative to the outer ends of the extended portions. The lens comprises an optic made from a flexible material combined with haptics capable of multiple flexions without breaking. The haptics are narrow adjacent the optic and wider at their outer ends. The haptics have wide and deep hinges adjacent the optic to better allow the hinges to “stretch” somewhat.

### CROSS REFERENCE TO RELATED APPLICATIONS

This application is a continuation-in-part of application Ser. No. 11/461,290 filed Jul. 31, 2006, the disclosure of which is incorporated by reference.

### BACKGROUND

Intraocular lenses have for many years had a design of a single optic with loops attached to the optic to center the lens and fixate it in the empty capsular bag of the human eye. In the mid '80s plate lenses were introduced, which comprised a silicone lens, 10.5 mm in length, with a 6 mm optic. These lenses could be folded but did not fixate well in the capsular bag, but resided in pockets between the anterior and posterior capsules. The first foldable lenses were all made of silicone. In the mid 1990s an acrylic material was introduced as the optic of lenses. The acrylic lens comprised a biconvex optic with a straight edge into which were inserted loops to center the lens in the eye and fixate it within the capsular bag.

Recently accommodating intraocular lenses have been introduced to the market, which generally are modified plate haptic lenses. A plate haptic lens may be referred to as an intraocular lens having two or more plate haptics joined to the optic.

Flexible acrylic material has gained significant popularity among ophthalmic surgeons. In 2003 more than 50% of the intraocular lenses implanted had acrylic optics. Hydrogel lenses have also been introduced. Both the acrylic and hydrogel materials are incapable of multiple flexions without fracturing.

The advent of an accommodating lens which functions by moving along the axis of the eye by repeated flexions somewhat limited the materials from which the lens could be made. Silicone is the ideal material, since it is flexible and can be bent probably several million times without showing any damage. Additionally a groove or hinge can be placed across the plate adjacent to the optic as part of the lens design to facilitate movement of the optic relative to the outer ends of the haptics. On the other hand, acrylic material fractures if it is repeatedly flexed.

### SUMMARY OF THE INVENTION

According to a preferred embodiment of this invention, an accommodating lens comprises a lens with a flexible solid optic attached to which are two extended portions which probably are plate haptics capable of multiple flexions without breaking, preferably along with fixation and centration features at their distal ends. There is a wide or narrow hinge or groove across the extended portions adjacent to the optic to facilitate the anterior and posterior movement of the optic relative to the outer ends of the extended portions. The wide base of the flexible hinge allows stretching of the base of the hinge with ciliary muscle contraction and an increase of vitreous cavity pressure thus allowing additional anterior movement of the optic relative to the outer ends of the haptics.

Accordingly, features of the present invention are to provide an improved form of accommodating lens.

### BRIEF DESCRIPTION OF THE DRAWINGS

FIG. 1 is a front elevational view of a preferred embodiment of the present invention.

FIG. 2 is a side view.

FIG. 3 is a detail view of a hinge.

According to the present invention the optic is of a foldable, flexible silicone, acrylic or hydrogel material and the haptic plates are of a foldable material that will withstand multiple foldings without damage, e.g., silicone. Preferably, the end of the plate haptics essentially have T-shaped fixation devices and are hinged to the optic.

### DESCRIPTION OF THE PREFERRED EMBODIMENT

Turning now to the Figures, a preferred embodiment is illustrated in detail comprising an intraocular lens 1 formed as a flexible solid optic 2 preferably made of silicon, and flexible extending portions 4 of any suitable form but preferably triangular plate haptics which are capable of multiple flexations without damage and formed, for example, of silicone. The optic 2 and haptics 4 preferably are uniplanar, and two haptics 4 extend distally from opposite sides of the optic 2. Fixation and centration fingers 6 are provided at the distal ends of the haptics 4.

A typical length for the lens 11 is 10.5-11.5 mm, and the optic 2 typically is a 4.5-5.0 mm diameter optic. The fingers 6 preferably are approximately 5.0 mm wide and comprise four-point fixation loops that extend distally when the lens is put into any insertion cartridge. The ends 8 have a slightly different configuration and aid in indicating to the surgeon that the lens is right side up with the hinges in a proper position.

Importantly, the haptics 4 have a triangular shape, narrower adjacent the optic, and wider at the outer ends. Hinges 10 are provided between the haptics 4 and the outer periphery of the optic 2, and it is particularly desirable to have a wide elastic base 10 to the hinge to allow the optic 2 to move forward more by stretching of the thin hinge base with the increase in vitreous cavity pressure which allows more anterior movement than in current designs such as for example that shown in U.S. Pat. No. 6,387,126. A typical hinge width 11 is 0.4-6.0 mm, and preferably with a hinge base width longitudinally as indicated by arrow 12 of 0.1 to 0.8 mm and preferably 0.5 mm, and a thickness range as indicated by arrow 14 of 0.06-0.4 mm, and preferably 0.12 mm, as seen in FIG. 3. The wider hinge base stretches somewhat like an elastic band to facilitate greater anterior movement of the optic 2.

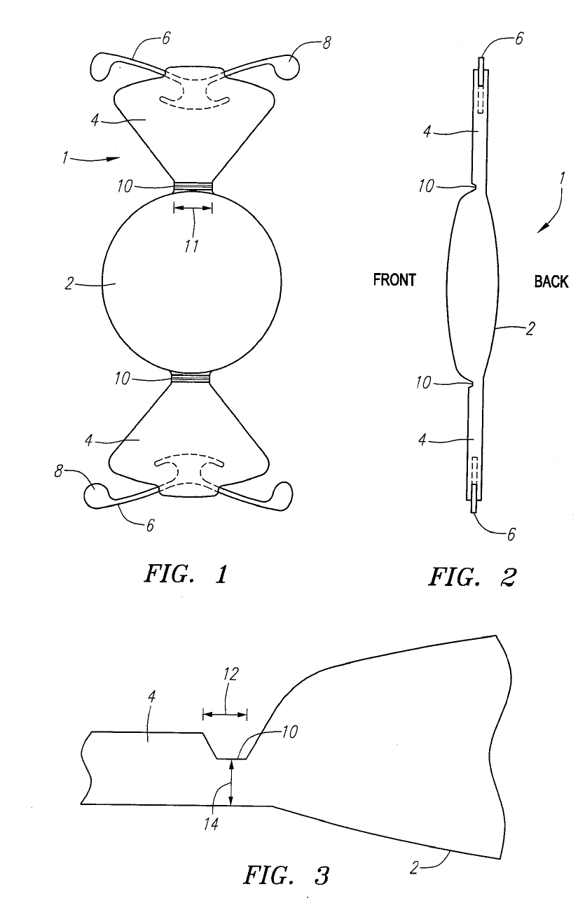

The hinges 10 are on the anterior side and the round end 8 of loops 6 on the right as seen in FIG. 1 indicates that the hinge is uppermost. End 8 is round. The wider loops 6 minimize the anterior vault of the lens for distance vision and therefore provide better distance vision.

Preferably the optic and plate haptics are silicone and the loops 6 are polyimide.

There can be a sharp edge around the posterior surface of the optic 2. To reduce the migration of cells across the posterior capsule of the lens post-operatively and thereby reduce the incidence of posterior capsular opacification and the necessity of YAG posterior capsulotomy.

As is well known in the art, the intraocular lens 1 such as that in the drawings is implanted in the capsular bag of the eye after removal of the natural lens. The lens is inserted into the capsular bag by a generally circular opening cut in the anterior capsular bag of the human lens and through a small opening in the cornea or sclera. The outer ends of the haptics 4, or loops 6, are positioned in the cul-de-sac of the capsular bag. The outer ends of the haptics, or the loops, are in close proximity with the bag cul-de-sac, and in the case of any form of loops, such as 6, the loops are deflected from the configuration. The ends or knobs of the loops are provided on the outer end portions of the loops 6 for improved securement in the capsular bag or cul-de-sac by engagement with fibrosis, which develops in the capsular bag following the surgical removal of the central portion of the anterior capsular bag.

The inner ends of the loops 6 may be either integrally formed from the same material as the haptics 4 or the loops may be of a separate material such as polyimide. The loops if formed of a separate material are molded into the terminal portions of the haptics 4 such that the flexible material of the loop 6 can extend by elasticity along the internal fixation member of the loop.

Accordingly, there has been shown and described a lens that ideally comprises a silicon optic and silicone haptic plates, loops that can be of a different material than the plate, and a fixation device at the end of each loop allowing for movement of the loops along the tunnel formed in the fusion of the anterior and posterior capsules of the human capsular bag and with wide hinges with a wide base that stretch like a rubber band.

Various changes, modifications, variations, and other uses and applications of the subject invention will become apparent to those skilled in the art after considering this specification together with the accompanying drawings and claims. All such changes, modifications, variations, and other uses of the applications which do not depart from the spirit and scope of the invention are intended to be covered by the claims which follow.

What is claimed is:

1. An accommodating intraocular lens comprising a flexible solid optic and attached flexible extended portions comprising plate haptics, designed such that the optic can move backward and forward relative to the outer ends of the extended portions and may assume a position such that the optic can be in front of, in the same plane or behind the outer ends of the haptics and can achieve accommodation by the optic moving forward toward the iris from a posterior to a more anterior or uniplanar position relative to the outer ends of the extending portions, and wherein the haptics are relatively narrow adjacent the optic and are wider distally, and the lens comprising hinges in the haptics adjacent the optic.

2. A lens according to claim 1 wherein the hinges are wide.

3. A lens according to claim 1 wherein the hinges are deep.

4. A lens according to claim 1 wherein the hinges are wide and deep.

5. A lens according to claim 1 wherein one or more fixation devices are on the ends of the extended portions.

6. A lens according to claim 1 where the optic is silicone.

7. A lens according to claim 1 where the haptics are silicone.

8. A lens according to claim 1 where the extended portions include loops and fixation devices of polyimide.

9. A lens according to claim 5 where the loops have a fixation element of a different shape on their proximal ends to enhance centration and fixation of the lens within the capsular bag.

10. A lens according to claim 1 where the optic size is from 3.5 to 8 mm.

11. A lens according to claim 1 wherein the hinges are wide at their base allowing stretching of the base with an increase in vitreous cavity pressure.
